(19) United States
(12) Plant Patent Application Publication
(10) Pub. No.: US 2016/0135350 P1
(43) Pub. Date: May 12, 2016

(54) Westringia hybrid plant named Wes08

(50) ;

(71) Applicant: Graham Brown, Cobbitty (AU)

(72) Inventor: Graham Brown, Cobbitty (AU)

(21) Appl. No.: 14/757,033

(22) Filed: Nov. 10, 2015

Related U.S. Application Data
(60) Provisional application No. 62/078,723, filed on Nov. 12, 2014.

(51) Int. Cl. A01H 5/00

(52) U.S. Cl. PLT/226

(57) ABSTRACT

‘WES08’ is a distinctive variety of Westringia hybrid which is characterized by the combination of a rotund shape, very short plant height, a high degree of branching and greyed-green foliage.

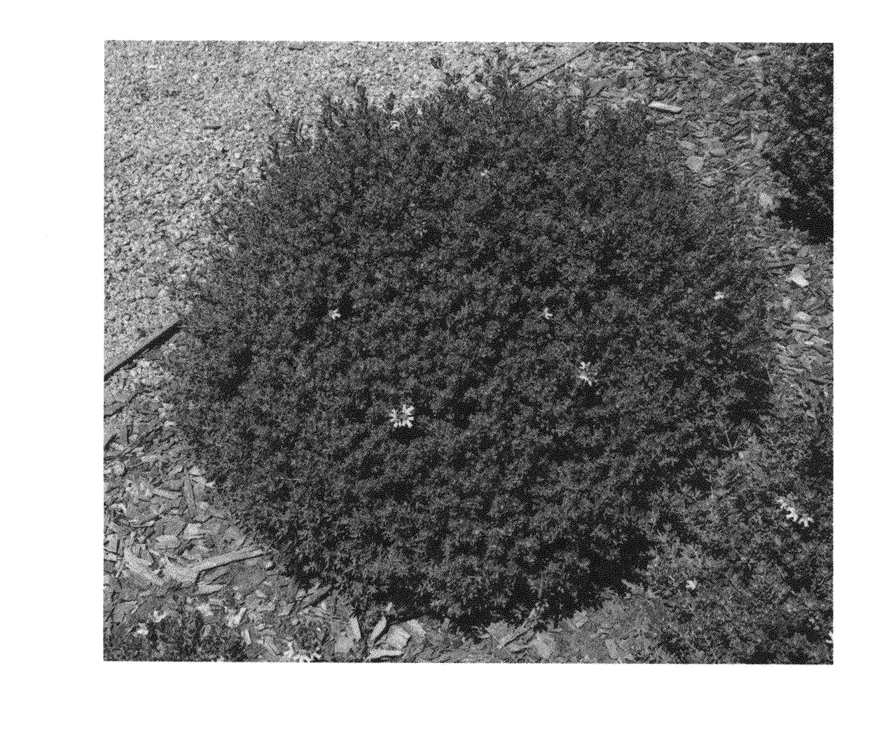

### CROSS REFERENCE TO RELATED APPLICATIONS

This application claims the benefit of U.S. Provisional Application No. 62/078,723 filed Nov. 12, 2014, which is herein incorporated by reference.

### LATIN NAME OF THE GENUS AND SPECIES

The Latin name of the genus and species of the novel variety disclosed herein is Westringia hybrid.

### VARIETY DENOMINATION

The inventive variety of Westringia hybrid disclosed herein has been given the variety denomination ‘WES08’.

### BACKGROUND OF THE INVENTION

The present invention relates to a new and distinct perennial variety of Westringia fruticosa, which has been given the variety denomination of ‘WES08’. Its market class is that of a shrub. ‘WES08’ is intended for use in general landscaping and as a decorative plant.

Parentage: The Westringia hybrid variety ‘WES08’ is a seedling selection that resulted from the open pollination of a male parent breeding line of Westringia hybrid (unpatented), developed from Westringia hybrid ‘WES01’ (unpatented), and female parent Westringia hybrid ‘WES01’ (unpatented) at a commercial plant breeding facility in Cobbitty, NSW Australia. An isolated breeding block of the parents was established in 2005 and, in November 2007, seed was harvested from female parent, Westringia hybrid ‘WES01’. Said seed was subsequently sown and March of 2008 and resulting plants were transplanted into raised field beds in September of 2008 for further observations. Progeny were further observed for desired commercial characteristics and, in September 2010, one plant was selected for its rotund shape, very short plant height, a high degree of branching and greyed-green foliage. The cultivar was given the denomination of ‘WES08’.

Asexual Reproduction: ‘WES08’ was first propagated asexually by semi-hardwood cuttings in Cobbitty, New South Wales, Australia in September of 2010 and has since been asexually propagated by vegetative cuttings through several successive generations. The distinctive characteristics of the inventive ‘WES08’ variety are stable from generation to generation; clones of the variety produced by asexual reproduction maintain the distinguishing characteristics of the original plant.

### SUMMARY OF THE INVENTION

‘WES08’ is a distinctive variety of Westringia hybrid which is characterized by the combination of a rotund shape, very short plant height, a high degree of branching and greyed-green foliage.

### BRIEF DESCRIPTION OF THE FIGURES

The FIGURE shows, as nearly true as it is reasonably possible to make the same in color illustrations of this type, an exemplary 18 month old ‘WES08’ plant in the landscape.

### BOTANICAL DESCRIPTION OF THE PLANT

The following is a detailed botanical description of a new and distinct variety of a Westringia hybrid ornamental plant known as ‘WES08’. Unless indicated otherwise, the descriptions disclosed herein are based upon observations made from an 18 month old ‘WES08’ plant, established in a full-sun landscape in Clarendon, New South Wales, Australia. The observed plant was maintained with granular slow release fertilizer and regularly watered with overhead irrigation. No pest and disease measures were taken.

Those skilled in the art will appreciate that certain characteristics will vary with older or, conversely, younger plants. ‘WES08’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 2001 edition. Note that generic color descriptions such as ‘white’ do not exist in the R.H.S. charts and the corresponding R.H.S. colors are quoted.
Plant description:
   Plant habit.—Shrub; very short height and rotund in shape, with a dense growth habit.
   Height.—600 mm.
   Width.—800 mm.
   Bloom period.—Spring and sporadically at other times of year, depending on climate.
   Hardiness.—USDA Zone 9 to 11.
   Propagation.—Propagation is accomplished using semi-hard wood cuttings. Roots well without rooting compound or hormone.
   Time to develop roots.—approximately 21 days at 20 degrees Celsius.
   Crop time.—From approximately five to eight months are needed to produce a well rooted 15 cm nursery pot, starting from a rooted cutting. Crop time varies with location and production practices.
   Pest and disease susceptibility or resistance.—Good resistance to crown and root rotting fungal pathogens.
   Cultural tolerances.—Westringia is commonly known to perform well in sandy soils, but also tolerates heavy, clay-type soils well. ‘WES08’ can also be grown in a relatively wide pH range and is also tolerant of above-normal salt concentration in the soil environment and is also tolerant of aerosol salt exposure. ‘WES08’ has good drought tolerance, once established.
Roots: The roots of ‘WES08’ are colored white and fleshy and thick, similar to other Westringia species and cultivars.
Stem:
   General habit.—Multiple main stems, basally branched.
   Stem strength.—Medium rigidity.
   Attitude.—Stems are generally 60 degrees from vertical.
   Stem cross-section.—Round with ridges.
   Stem color.—194A at 10 nodes below the active growing point.
   Stem dimensions.—7.0 mm in length with a diameter of 1.3 mm, at 10 nodes below the active growing point.
   Stem surface.—Rigid and pubescent.
   Internode length.—10 mm, at 10 nodes below the active growing point.
Foliage:
   Type.—Evergreen.
   Shape.—Elliptical, narrow.
   Division.—Simple.
   Apex.—Acute.
   Base.—Cuneate.
   Venation.—reticulate but not obvious.
   Vein color (adaxial surfaces).—indistinguishable from surrounding foliage.
   Vein color (abaxial surfaces).—indistinguishable from surrounding foliage.
   Margins.—Entire.
   Arrangement.—Whorled.
   Attachment.—Petiolate.
   Texture.—Leathery.
   Surfaces (adaxial surface).—Finely pubescent to glabrous.
   Surfaces (abaxial surface).—Lightly pubescent.
   Mature leaf dimensions.—Average length, 9 mm; width is 4 mm.
   Leaf color (adaxial surface).—137A.
   Leaf color (abaxial surface).—N138C.
   Petiole.—Approximately 1 mm in length; width is approximately 1 mm.
   Stipules.—absent.
Inflorescence: Flowers are sessile.
Bud: Obovoid in shape and rounded at the apex. Approximately 5 mm long and 2.5 mm wide prior to dehiscence. Color is N82B.
Flower:
   General.—Flowers are sessile, simple and persist for approximately 5 days. Self cleaning. Non fragrant.
   Flowers.—Small; Sympetalous, irregular with petals numbering five, width across the corolla measuring on average 12 mm; length of the perianth, from base of calyx to the furthest outstretched point of the corolla, measures 12 mm; color is N82B with brown spots, or “eyes”, present. Margins cilliate; all surfaces are pubescent.
   Calyx.—Synsepalous with five pointed calyx lobes; color is green 137D. The length of the calyx, including sepal lobes, is 5 mm.
Reproductive organs:
   Stamens.—4 and 2 staminodes.
   Stamen color.—N82B.
   Stamen dimensions.—About 4 mm in length and less than 1 mm wide.
   Attachment.—Adnate.
   Anther dimensions.—Bilobate; approximately 0.5 mm long.
   Anther color.—N82B and slightly translucent.
   Pollen.—Absent.
   Pistil.—One.
   Pistil dimensions.—6 mm in length and less than 1 mm wide.
   Pistil color.—N82B and slightly translucent.
   Ovary position.—Inferior.
Fruit and seed: Not observed.
Comparisons with parent plants: Plants of the new cultivar ‘WES08’ are similar to female parent in many horticultural characteristics. However, ‘WES08’ exhibits a very short plant height and a rotund shape whereas the female parent, ‘WES01’, exhibits a tall plant height of approximately 180 to 190 cm and an upright growth habit. ‘WES01’ is also shorter and more compact than the other Westringia breeding lines that were used in the initial open pollination in 2005.
Comparisons with other commercial Westringia: Plants of the new cultivar ‘WES08’ are comparable to the commercial varieties Westringia fruticosa ‘WES02’ (U.S. patent application Ser. No. 13/986,462) and Westringia fruticosa ‘WES04’ (U.S. Plant Pat. No. 25,675) in that all three varieties exhibit a rotund growth habit and relatively short plant height. However, ‘WES08’ exhibits a shorter plant height and smaller leaves compared to ‘WES02’ and ‘WES04’. Also, the flowers of ‘WES08’ are generally violet whereas the flowers of ‘WES04’ are generally white. Furthermore, the foliage of ‘WES08’ is lightly pubescent whereas the foliage of ‘WES04’ is highly pubescent.

That which is claimed is:

1. A new and distinct variety of Westringia hybrid plant named ‘WES08’, substantially as described and illustrated herein.
